SEQUENCE LISTING (4 sequences)

SEQ ID NO: 1 - protein, 12 aa, synthetic construct
GRKKRRQRRRPQ

SEQ ID NO: 2 - protein, 12 aa, synthetic construct
GGVPMSMRGGGK

SEQ ID NO: 3 - protein, 12 aa, synthetic construct
GGGPQGIAGQGK

SEQ ID NO: 4 - protein, 13 aa, synthetic construct
GGGMGPSGPWGGK

(19) United States
(12) Patent Application Publication
(10) Pub. No.: US 2025/0387341 A1
(43) Pub. Date: Dec. 25, 2025

(54) MICROFLUIDIC ASSEMBLY OF MITOCHONDRIA-LOADED MICROPARTICLES FOR ON-DEMAND DELIVERY

(71) Applicant: Georgia Tech Research Corporation, Atlanta, GA (US)

(72) Inventors: Johnna Temenoff, Atlanta, GA (US); Ryan Cree Miller, Atlanta, GA (US)

(21) Appl. No.: 19/246,121

(22) Filed: Jun. 23, 2025

Related U.S. Application Data
(60) Provisional application No. 63/662,582, filed on Jun. 21, 2024.

(51) Int. Cl. A61K 9/50; A61K 9/06; A61K 35/12; A61K 47/54; A61K 47/60; A61K 47/61; A61K 47/64; A61P 19/00; A61P 21/00

(52) U.S. Cl. CPC A61K 9/5031; A61K 9/06; A61K 35/12; A61K 47/548; A61K 47/60; A61K 47/61; A61K 47/645; A61P 19/00; A61P 21/00

(57) ABSTRACT

The present disclosure provides a composition for modifying the surface of mitochondria, comprising: a polymer backbone; a first chemical moiety conjugated to the polymer backbone, the chemical moiety comprising a peptide; and a second chemical moiety conjugated to the polymer backbone, the second chemical moiety comprising triphenylphosphine. The disclosure also provides a hydrogel comprising a plurality of hydrogel particles, each particle comprising a first polymer crosslinked with an enzyme cleavable peptide to form a crosslinked polymer and at least one mitochondria, wherein each hydrogel particle has an average diameter no greater than 100 microns. Methods of treating a disease and/or injury in a subject by administering the hydrogel are also provided.

### CROSS-REFERENCE TO RELATED APPLICATIONS

This application claims priority to U.S. Provisional Application No. 63/662,582, filed 21 Jun. 2024, which is hereby fully incorporated by reference in its entirety.

### SEQUENCE LISTING

The instant application contains a computer readable Sequence Listing which has been submitted electronically in XML format (“Sequence Listing XML”) and is hereby incorporated by reference in its entirety. The Sequence Listing XML, created on Jun. 23, 2025, is named 011529.114798.xml and is 4,357 bytes in Size.

### FIELD OF INVENTION

The present disclosure relates to mitochondrial delivery systems, and more particularly to microfluidic assembly of enzyme-responsive hydrogel microparticles containing surface-modified mitochondria for controlled release and enhanced cellular uptake.

### BACKGROUND

Mitochondria are essential organelles in eukaryotic cells, responsible for energy production through cellular respiration. These double-membrane-bound structures generate adenosine triphosphate (ATP), the primary energy currency of cells, through oxidative phosphorylation. In recent years, research has revealed that mitochondria play roles beyond energy production, including involvement in cell signaling, apoptosis regulation, and calcium homeostasis.

The transfer of mitochondria between cells has emerged as a topic of interest in cellular biology and regenerative medicine. This phenomenon, known as mitochondrial transfer, involves the movement of mitochondria from one cell to another. Studies have shown that mitochondrial transfer can occur naturally in various physiological and pathological contexts, potentially influencing cellular function and tissue repair processes.

Efforts to harness mitochondrial transfer for therapeutic purposes have gained attention in the scientific community. Researchers have explored methods to facilitate the delivery of exogenous mitochondria to target cells or tissues. However, developing effective delivery systems for mitochondria presents several challenges. These include maintaining mitochondrial viability during the delivery process, ensuring efficient uptake by recipient cells, and controlling the release of mitochondria at desired locations and times.

Hydrogels have been widely investigated as biomaterials for various biomedical applications, including drug delivery and tissue engineering. These three-dimensional networks of hydrophilic polymers can be designed to respond to specific stimuli, such as changes in pH, temperature, or the presence of certain enzymes. Enzyme-responsive hydrogels, in particular, have shown promise for controlled release applications in biological environments.

Microfluidic technologies have enabled the production of precisely controlled microparticles for diverse applications in biotechnology and medicine. These techniques allow for the generation of uniform particles with tunable sizes and compositions. The integration of microfluidic particle production with hydrogel materials and biologically active components offers potential for creating advanced delivery systems.

Surface modification of cellular components, including organelles like mitochondria, can influence their interactions with other cellular structures and potentially enhance their uptake by cells. Various approaches have been explored to modify the surface properties of biological entities, including the use of polymers and bioactive molecules.

As research in the fields of mitochondrial biology, biomaterials, and microfluidics continues to advance, there remains a need for innovative approaches to address the challenges associated with mitochondrial delivery for potential therapeutic applications.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a composition for modifying the surface of mitochondria is provided. The composition includes a polymer backbone, a first chemical moiety conjugated to the polymer backbone, and a second chemical moiety conjugated to the polymer backbone. The first chemical moiety comprises a peptide according to SEQ ID NO: 1. The second chemical moiety comprises triphenylphosphine.

According to other aspects of the present disclosure, the composition may include one or more of the following features. The polymer backbone may be an inert polymer backbone. The polymer backbone may be a polysaccharide. The polysaccharide may be a dextran.

According to another aspect of the present disclosure, a hydrogel is provided. The hydrogel includes a plurality of hydrogel particles. Each of the hydrogel particles comprises a first polymer crosslinked with an enzyme cleavable peptide to form a crosslinked polymer, and at least one mitochondria. Each hydrogel particle in the plurality of hydrogel particles has an average diameter no greater than 100 microns.

According to other aspects of the present disclosure, the hydrogel may include one or more of the following features. The first polymer may be polyethylene glycol. The enzyme cleavable peptide may be selected from the group consisting of: SEQ ID NO: 2; SEQ ID NO: 3; and SEQ ID NO: 4. The hydrogel may further comprise a second polymer. The second polymer may be poly(ethylene glycol) diacrylate (PEGDA). A ratio of the second polymer to the crosslinked polymer may be no greater than 1:1 mol %. A surface of the at least one mitochondria may be modified with the composition comprising the polymer backbone, the first chemical moiety, and the second chemical moiety. The polymer backbone of the composition modifying the surface of the at least one mitochondria may be a polysaccharide. The polysaccharide may be a dextran.

According to another aspect of the present disclosure, a method of treating a disease and/or injury in a subject is provided. The method includes administering, to the subject, the hydrogel comprising the plurality of hydrogel particles.

According to other aspects of the present disclosure, the method may include one or more of the following features. The disease and/or injury may be a musculoskeletal disease or injury. The musculoskeletal disease or injury may be selected from the group consisting of: muscle injury, bone injury, and cartilage injury. Administering may comprise injecting the hydrogel into a site of the disease and/or injury. The hydrogel may be administered in combination with a cell therapy. The cell therapy may comprise use of any tissue-derived cell, or blood-derived cell, such as stem cells (e.g., mesenchymal stem cells). Subsequent to administering, to the subject, the hydrogel, the hydrogel may react with matrix metalloproteinases produced by the subject as a result of the disease and/or injury to cause degradation of at least a portion of the hydrogel and release of the at least one mitochondria.

The foregoing general description of the illustrative embodiments and the following detailed description thereof are merely exemplary aspects of the teachings of this disclosure and are not restrictive. These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF FIGURES

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A illustrates a system diagram of functionalized mitochondria and a hydrogel delivery system, according to aspects of the present disclosure.

FIG. 1B provides a plot of cumulative mitochondria release, according to aspects of the present disclosure.

FIG. 1C depicts images and graphs showing normalized fold changes in mitochondria uptake in cells, according to aspects of the present disclosure.

FIG. 1D shows a graph of differentiation and fusion index scores for experimental conditions, according to aspects of the present disclosure.

FIG. 1E provides images of alizarin red stained MC3T3-E1 cells and a graph of absorbance measurements over time for multiple experimental conditions, according to aspects of the present disclosure.

FIG. 2A depicts a system diagram showing functionalized mitochondria and their cellular uptake mechanism, according to aspects of the present disclosure.

FIG. 2B illustrates enzyme-responsive peptide sequences and a hydrogel microparticle structure, according to aspects of the present disclosure.

FIGS. 3A-3C show microscopy images and data comparing mitochondrial uptake in C2C12 cells under different treatments, according to aspects of the present disclosure.

FIGS. 3D-3F depict microscopy images and data comparing mitochondria uptake in MC3T3-E1 cells under different treatments, according to aspects of the present disclosure.

FIGS. 3G-3K illustrate graphs and microscopy images showing mitochondrial uptake and analysis data, according to aspects of the present disclosure.

FIGS. 4A-4C depict a microfluidic droplet generation system for producing mitochondria-loaded microparticles, according to aspects of the present disclosure.

FIGS. 4D-4F show graphs of experimental data related to collagenase concentration, PEG-VPM: PEGDA ratio, and microparticle diameter effects on mitochondria release, according to aspects of the present disclosure.

FIGS. 5A-5F illustrate chemical structures and experimental data related to mitochondrial release and degradation kinetics, according to aspects of the present disclosure.

FIGS. 6A-6C depict mitochondrial polarization states and related experimental data, according to aspects of the present disclosure.

FIGS. 6D-6E show graphs of oxygen consumption rate measurements and analysis, according to aspects of the present disclosure.

FIGS. 7A-7B illustrate a timeline and microscopy images related to mitochondrial transfer and differentiation experiments, according to aspects of the present disclosure.

FIGS. 7C-7E depict a bar graph and microscopy images comparing mitochondria uptake in C2C12 cells, according to aspects of the present disclosure.

FIGS. 7F-7I show graphs of differentiation and fusion metrics for cells under various treatment conditions, according to aspects of the present disclosure.

FIGS. 8A-8B depict microscopy images and a timeline diagram showing proliferation and differentiation phases with associated measurements, according to aspects of the present disclosure.

FIG. 8C illustrates a bar graph comparing normalized fold changes in mitochondrial uptake between treatments, according to aspects of the present disclosure.

FIG. 8D provides representative brightfield images of MC3T3-E1 cells at 7 and 14 days of differentiation, according to aspects of the present disclosure.

FIG. 8E shows a graph of calcium deposition measurements over time for different experimental conditions, according to aspects of the present disclosure.

FIG. 8F depicts a graph of alkaline phosphatase activity measurements over time for various experimental conditions, according to aspects of the present disclosure.

### DETAILED DESCRIPTION

Although preferred exemplary embodiments of the disclosure are explained in detail, it is to be understood that other exemplary embodiments are contemplated. Accordingly, it is not intended that the disclosure is limited in its scope to the details of construction and arrangement of components set forth in the following description or illustrated in the drawings. The disclosure is capable of other exemplary embodiments and of being practiced or carried out in various ways. Also, in describing the preferred exemplary embodiments, specific terminology will be resorted to for the sake of clarity.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used in the specification and the appended claims, the singular forms “a,” “an” and “the” include plural referents unless the context clearly dictates otherwise.

Also, in describing the preferred exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

Reference will now be made in detail to exemplary embodiments of the disclosed technology, examples of which are illustrated in the accompanying drawings and disclosed herein.

The present disclosure relates to systems and methods for microfluidic assembly of mitochondria-loaded microparticles for on-demand delivery. In some cases, the systems and methods may include functionalized mitochondria encapsulated within enzyme-responsive hydrogel microparticles. The functionalized mitochondria may comprise surface modifications to enhance cellular uptake. The hydrogel microparticles may be designed to degrade in response to specific enzymes, allowing controlled release of the encapsulated mitochondria.

In some cases, the systems and methods described herein may be used for treating diseases and/or injuries in a subject. The microparticles containing functionalized mitochondria may be administered to a subject to deliver mitochondria to targeted tissues or cells. In some cases, the disease and/or injury treated may be a musculoskeletal disease or injury. The musculoskeletal diseases or injuries that may be treated using the disclosed systems and methods may include muscle injuries, bone injuries, and cartilage injuries.

The microfluidic assembly approach allows for precise control over microparticle size, composition, and mitochondrial loading. The enzyme-responsive nature of the hydrogel microparticles may enable on-demand release of mitochondria in response to disease- or injury-associated enzymes present at a treatment site. The functionalized mitochondria released from the microparticles may then be taken up by cells to potentially enhance cellular function and tissue regeneration.

The systems and methods described herein may have applications in regenerative medicine, tissue engineering, and treatment of various diseases and injuries where mitochondrial transfer could provide therapeutic benefits. The microfluidic assembly process may allow for scalable production of mitochondria-loaded microparticles with tunable properties for different applications.

The composition for modifying the surface of mitochondria may include a polymer backbone conjugated with two chemical moieties. In some cases, the polymer backbone may be any inert polymer. The polymer backbone may be a polysaccharide. In some cases, the polysaccharide may be a dextran.

The first chemical moiety conjugated to the polymer backbone may comprise a peptide according to SEQ ID NO: 1. This peptide may be a transactivator of transcription (TAT) peptide. In some cases, the TAT-peptide conjugation to the polymer backbone may be 10%.

The second chemical moiety conjugated to the polymer backbone may comprise triphenylphosphine (TPP). In some cases, the TPP conjugation to the polymer backbone may be 40%.

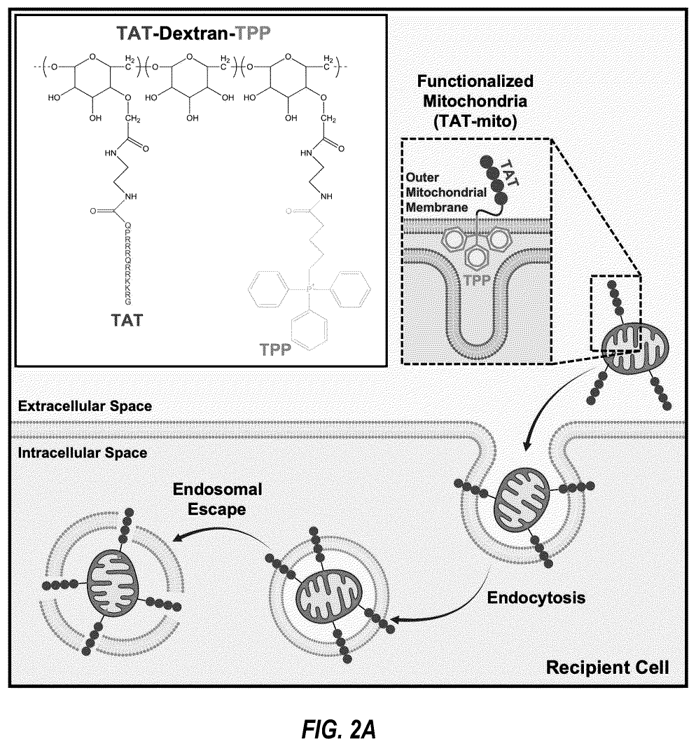

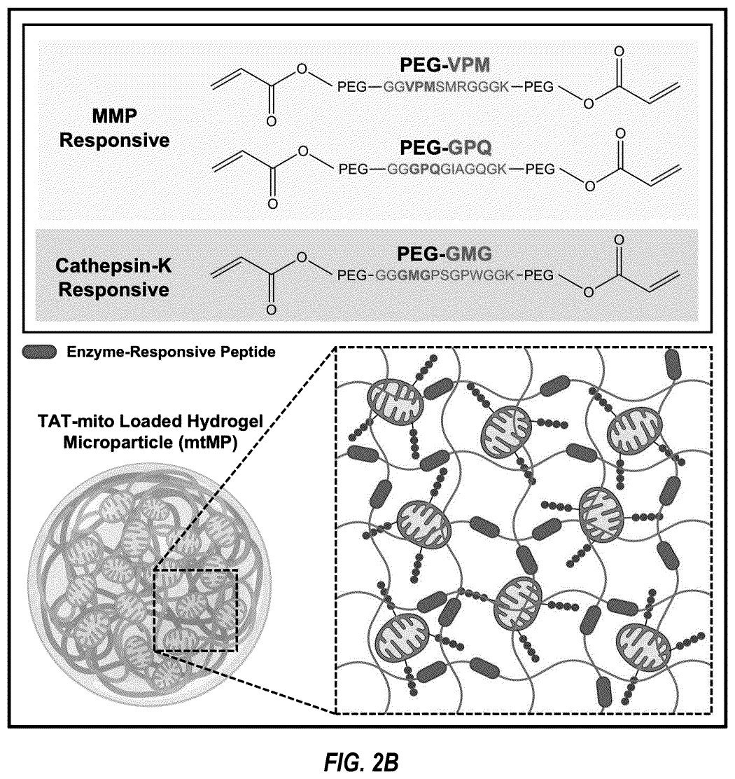

FIG. 2A illustrates a schematic of the composition for modifying the surface of mitochondria. The figure shows the molecular structure of the TAT-Dextran-TPP conjugate, with the dextran polymer backbone and the attached TAT peptide and TPP moieties.

The TAT peptide component may facilitate cellular uptake of the modified mitochondria through interactions with cell membranes. The TPP component may help anchor the polymer to the mitochondrial surface due to its lipophilic properties and positive charge.

In some cases, the mitochondria to be modified with this composition may be derived from mesenchymal stem cells. The mitochondria may be pre-stained with MitoTracker dyes prior to surface modification to allow for tracking and visualization in subsequent experiments or applications.

This composition may allow for surface modification of mitochondria to enhance their cellular uptake and delivery capabilities. The inert polymer backbone may provide a stable scaffold for attaching the functional moieties while minimizing potential interference with mitochondrial function.

The hydrogel microparticles may comprise a plurality of hydrogel particles. In some cases, each hydrogel particle in the plurality of hydrogel particles may have an average diameter no greater than 100 microns. FIG. 2B illustrates a schematic diagram of an enzyme-responsive hydrogel microparticle structure.

Each hydrogel particle may comprise a first polymer crosslinked with an enzyme cleavable peptide to form a crosslinked polymer. In some cases, the first polymer may be polyethylene glycol. The enzyme cleavable peptide may be selected from the group consisting of: SEQ ID NO: 2; SEQ ID NO: 3; and SEQ ID NO: 4. As shown in FIG. 2B, these peptide sequences may include PEG-VPM, PEG-GPQ, and PEG-GMG.

The hydrogel may further comprise a second polymer. In some cases, the second polymer may be poly(ethylene glycol) diacrylate (PEGDA). In some embodiments, a ratio of the second polymer to the crosslinked polymer may be no greater than 1:1 mol %. In some cases, the hydrogel may contain 10 wt % poly(ethylene glycol).

Each hydrogel particle may comprise at least one mitochondria. In some cases, a surface of the at least one mitochondria may be modified with the composition described previously for modifying mitochondrial surfaces. The hydrogel microparticles may contain varying concentrations of mitochondria protein, e.g., 20 μg/mL.

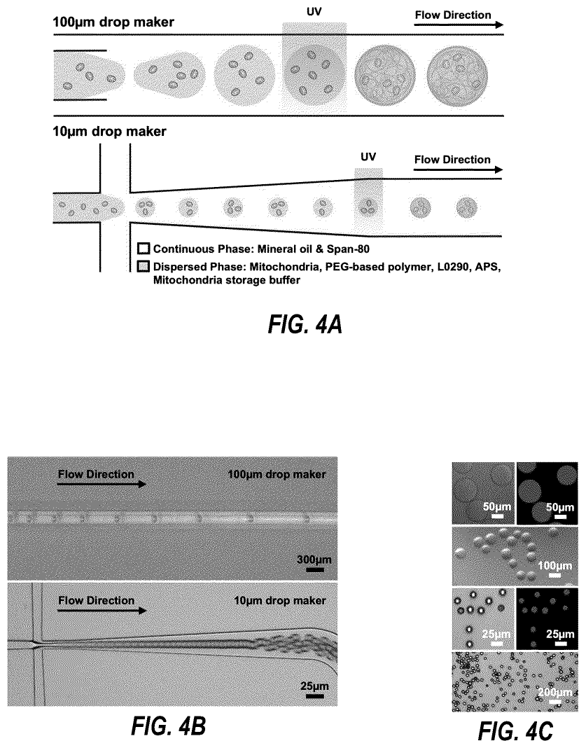

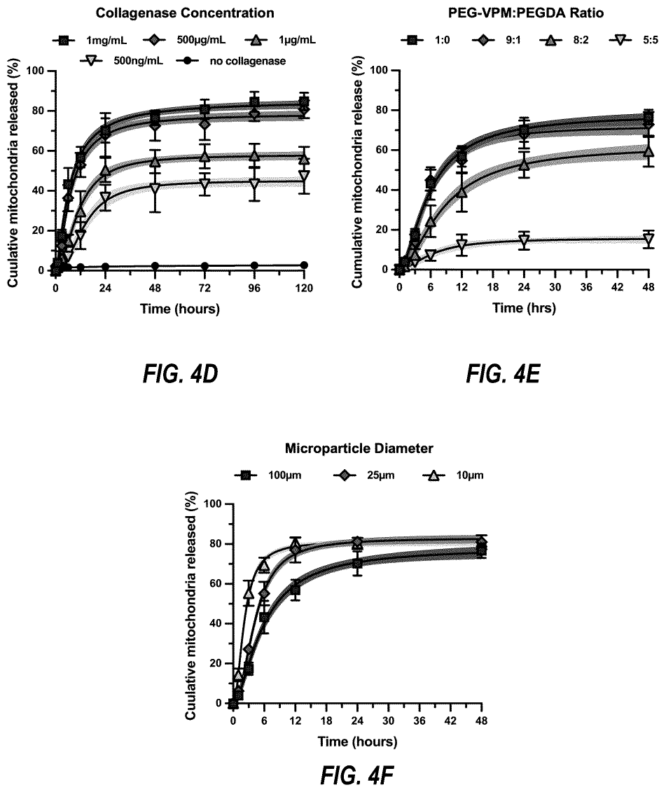

FIGS. 4A-C illustrates a microfluidic droplet generation system for producing mitochondria-loaded microparticles. The system demonstrates controlled formation of uniform droplets with precise size control through different channel dimensions. The scale markers in the images illustrate exemplary ranges of hydrogel microparticle sizes that may be produced using the microfluidic system.

In some cases, the hydrogel microparticles may have diameters of 10 μm, 25 μm, or 90 μm, among others. The microfluidic assembly approach may allow for precise control over microparticle size, composition, and mitochondrial loading.

The enzyme-responsive nature of the hydrogel microparticles may enable on-demand release of mitochondria in response to specific enzymes. This controlled release mechanism may be useful for targeted delivery of mitochondria in various applications.

The microfluidic assembly process may be used to create mitochondria-loaded microparticles. FIG. 4A, FIG. 4B, and FIG. 4C illustrate a microfluidic droplet generation system for producing the mitochondria-loaded microparticles. In some cases, the continuous phase may consist of mineral oil and Span-80. The dispersed phase may contain mitochondria, PEG-based polymer, L0290, APS, and mitochondria storage buffer.

In the 100 μm drop maker configuration, the system may demonstrate a sequence of droplet formation and UV crosslinking. The flow direction may be from left to right. The diagram shows the progressive formation of spherical droplets containing the dispersed phase. These droplets may be exposed to UV light at a specific point in the flow path. After UV exposure, the droplets may maintain their spherical structure as they continue flowing through the channel.

The 10 μm drop maker configuration may demonstrate a similar process but at a smaller scale. In some cases, this configuration may utilize a T-junction where the dispersed phase meets the continuous phase, creating smaller droplets. The flow direction may again be from left to right, with UV exposure occurring at a defined point along the channel. The resulting droplets may be significantly smaller than those produced in the 100 μm system.

The UV exposure region may be marked by a band across both channels, showing where crosslinking occurs in the process. This microfluidic assembly approach may allow for controlled formation of uniform droplets with precise size control through different channel dimensions.

In some cases, the hydrogel microparticles may be formed using this droplet microfluidics technique. The microfluidic assembly process may enable precise control over microparticle size, composition, and mitochondrial loading. The resulting mitochondria-loaded microparticles may have diameters ranging from 10 μm to 100 μm, among others, depending on the specific droplet maker configuration used.

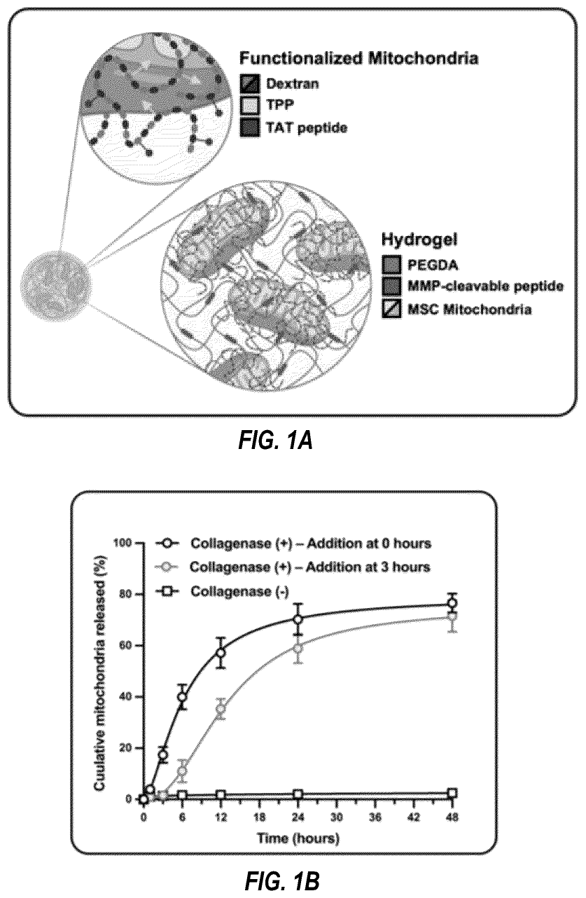

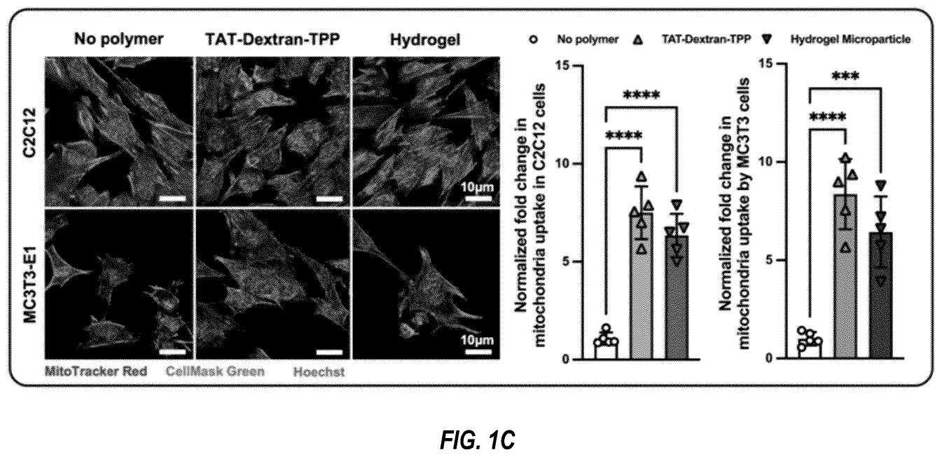

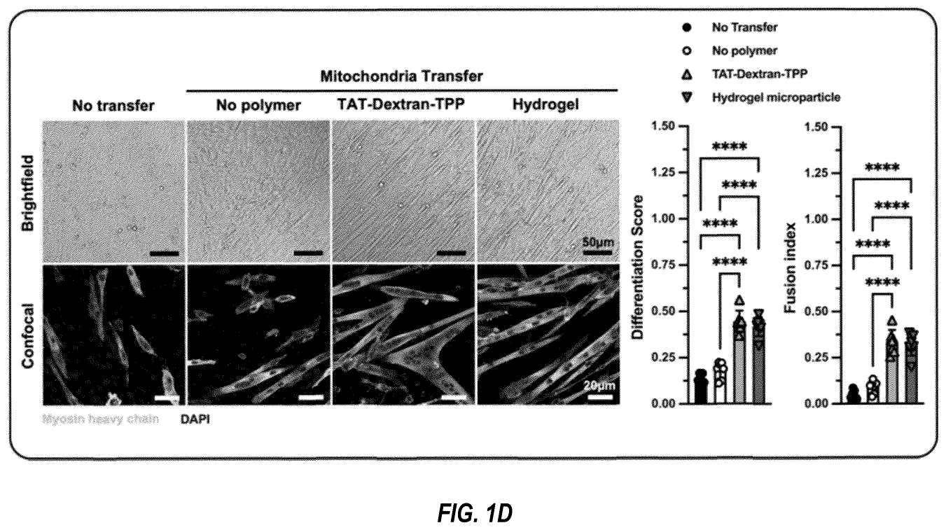

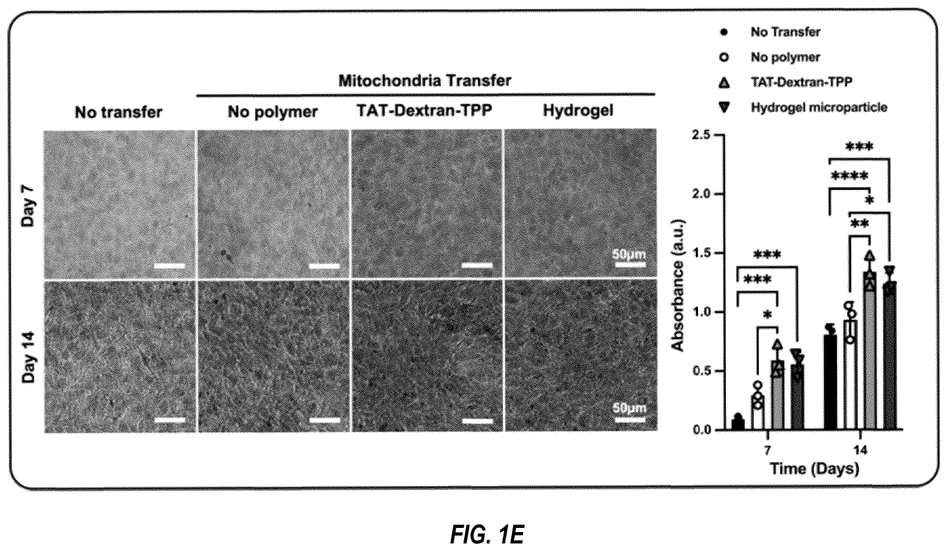

The mitochondrial uptake process may involve TAT-peptide facilitated endocytosis. FIG. 1A illustrates a system diagram showing functionalized mitochondria with TAT-peptide surface modifications. The TAT-peptide may enable enhanced cellular uptake of the modified mitochondria.

FIG. 1C depicts normalized fold changes in mitochondria uptake for different cell types. The graph demonstrates increased mitochondrial uptake with TAT-dextran-TPP functionalization compared to polymer-free conditions. In some cases, the TAT-dextran-TPP functionalization may result in a 7.5-fold increase in mitochondrial uptake for C2C12 cells and an 8.4-fold increase for MC3T3-E1 cells.

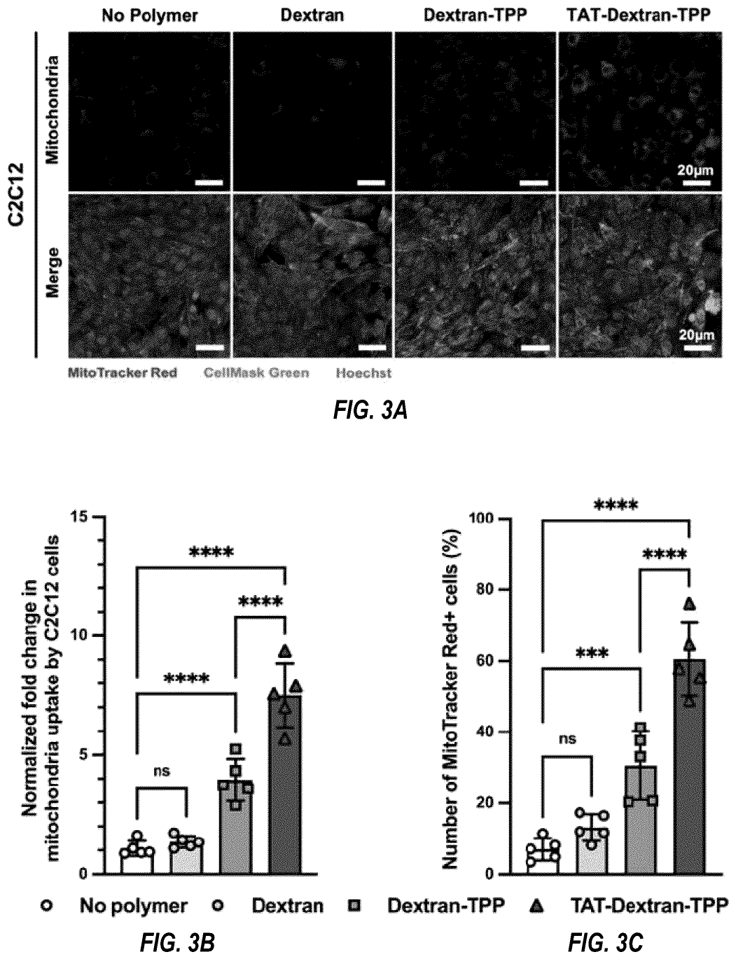

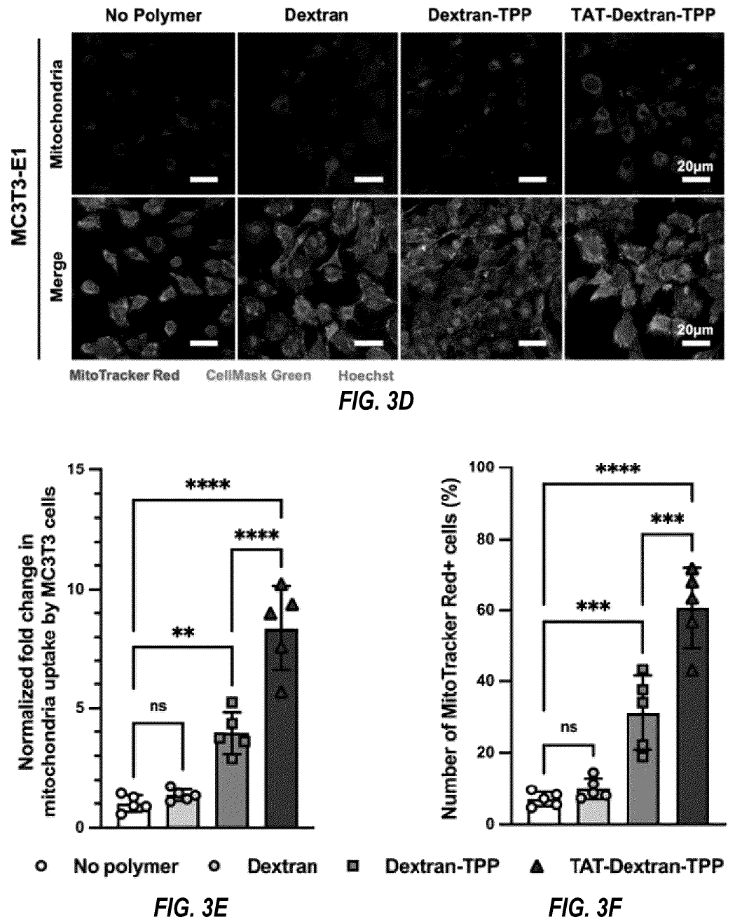

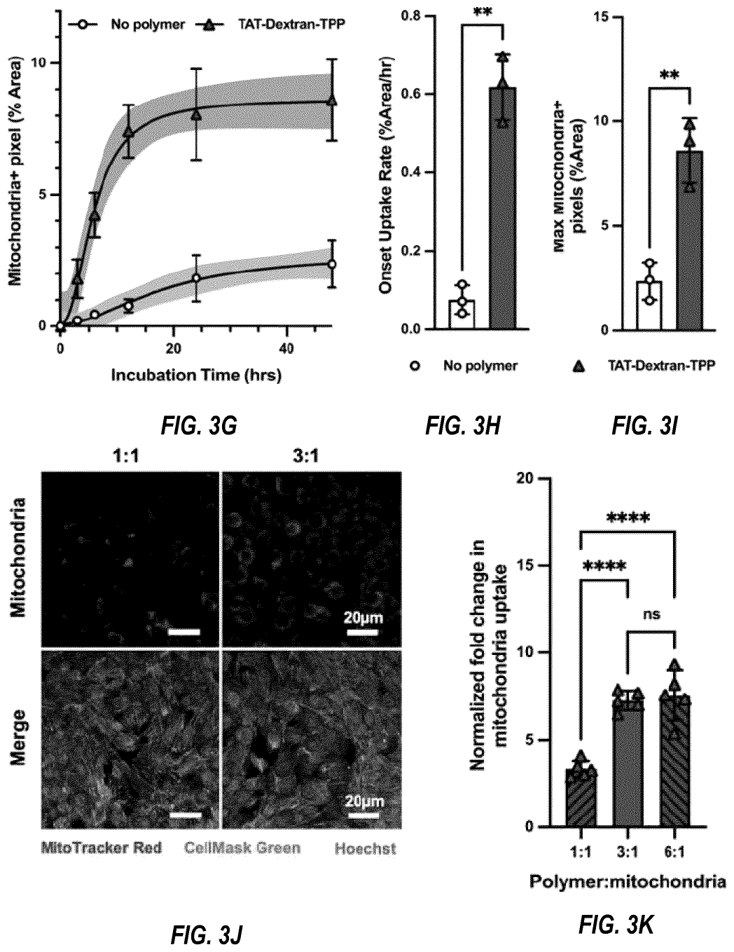

FIGS. 3G-3K show mitochondrial uptake kinetics and analysis data. The graphs illustrate how TAT-dextran-TPP functionalization may enhance the rate and extent of mitochondrial uptake compared to non-functionalized mitochondria. In some cases, the onset uptake rate for TAT-dextran-TPP functionalized mitochondria may be significantly higher than for non-functionalized mitochondria.

The controlled release of mitochondria from hydrogel microparticles may occur through enzyme-responsive mechanisms. FIGS. 4D-4F depict cumulative mitochondria release percentages under various conditions. FIG. 4D shows how different collagenase concentrations may affect mitochondria release over time. In some cases, higher collagenase concentrations may result in greater mitochondria release from the hydrogel microparticles.

FIG. 4E illustrates how varying ratios of PEG-VPM to PEGDA in the hydrogel composition may influence mitochondria release kinetics. The graph demonstrates that hydrogel microparticles with higher PEG-VPM content may exhibit increased mitochondria release over time. This relationship between hydrogel composition and release kinetics may allow for tuning of the release profile based on specific application requirements.

FIG. 4F shows how microparticle diameter may affect mitochondria release. The graph compares release profiles for 100 μm, 25 μm, and 10 μm diameter hydrogel microparticles. In some cases, smaller diameter microparticles may exhibit faster initial release rates compared to larger microparticles.

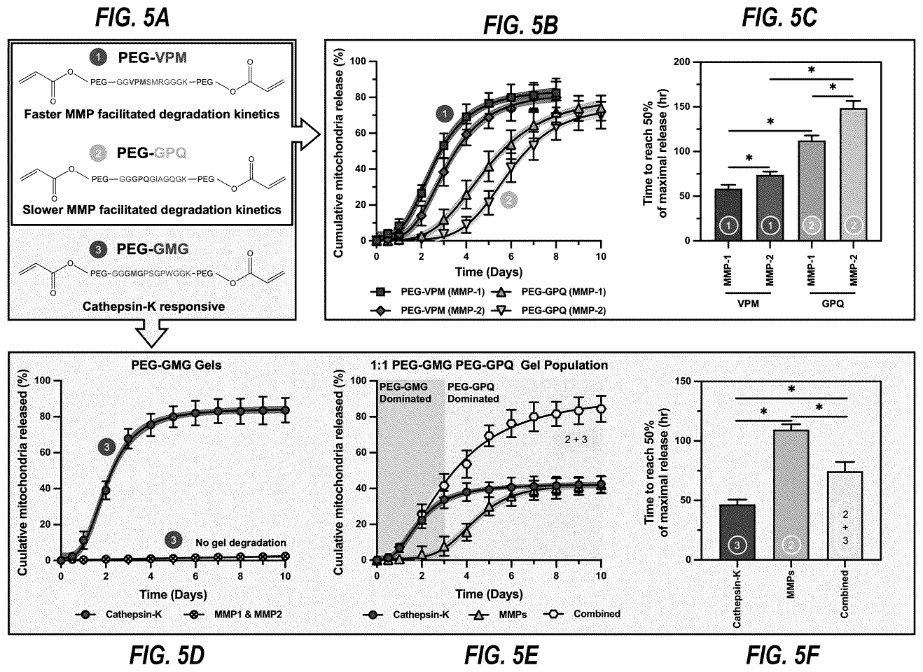

FIGS. 5A-5F provide additional data on mitochondrial release and degradation kinetics for different hydrogel compositions. The graphs show cumulative mitochondrial release percentages over time for hydrogel microparticles containing different enzyme-cleavable peptides. In some cases, hydrogel microparticles formed with PEG-VPM may exhibit faster release kinetics compared to those formed with PEG-GPQ when exposed to matrix metalloproteinases (MMPs).

The mitochondria released from the hydrogel microparticles may maintain a significant portion of their functionality. In some cases, the mitochondria may maintain 87% of their polarization after release from the hydrogel microparticles. This preservation of mitochondrial polarization may indicate that the encapsulation and release processes do not substantially impair mitochondrial function.

The enzyme-responsive nature of the hydrogel microparticles may allow for controlled mitochondria release in response to specific enzymes present at a treatment site. In some cases, the hydrogel microparticles may release mitochondria in response to specific concentrations of collagenase or MMPs. This targeted release mechanism may enable localized delivery of mitochondria in response to disease- or injury-associated enzyme activity.

The release kinetics of mitochondria from the hydrogel microparticles may be tuned by adjusting the composition of the hydrogel. In some cases, the hydrogel microparticles may be formed with different ratios of enzyme-cleavable peptides to control release kinetics. This ability to modulate release profiles may allow for customization of mitochondrial delivery based on specific therapeutic requirements or tissue environments.

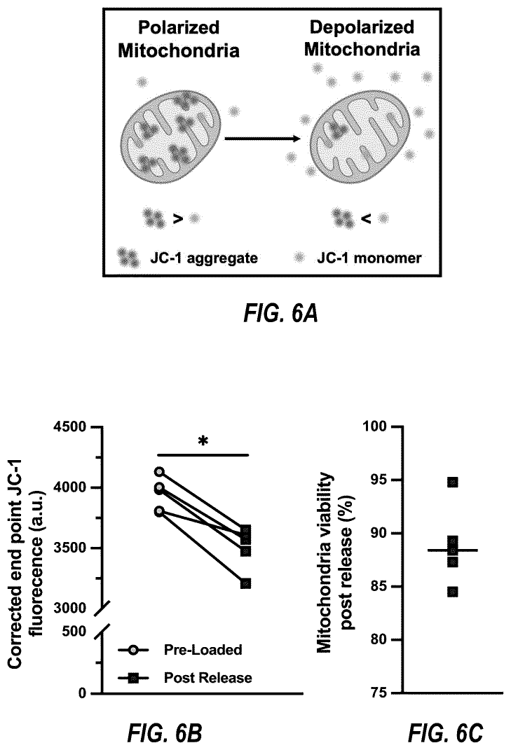

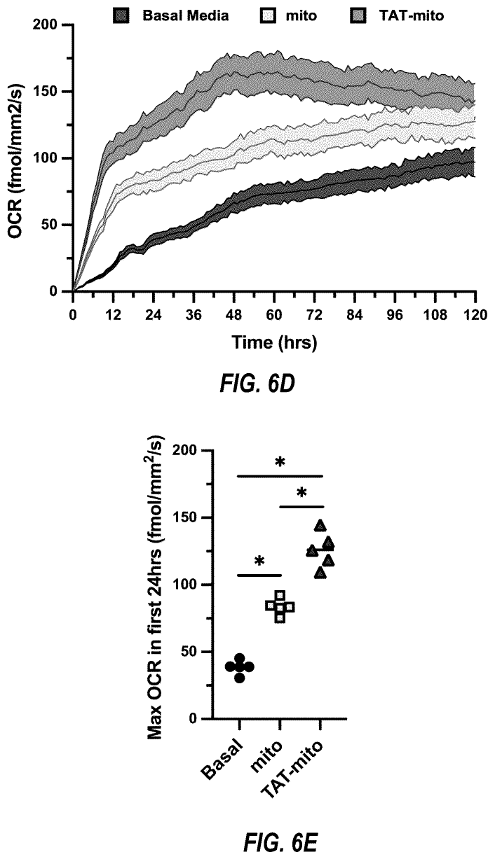

The mitochondrial transfer facilitated by the systems and methods described herein may result in various biological effects in recipient cells. In some cases, the transfer of mitochondria may lead to increased oxygen consumption rates in the recipient cells. FIGS. 6D-6E illustrate oxygen consumption rate (OCR) measurements for different experimental conditions. The graphs show that cells receiving TAT-functionalized mitochondria (TAT-mito) may exhibit higher OCR values compared to control conditions. This increased OCR may indicate enhanced metabolic activity in the recipient cells following mitochondrial transfer.

The transfer of mitochondria may also enhance differentiation processes in recipient cells. FIG. 1D depicts differentiation and fusion index scores for various experimental conditions in muscle cells. The graph demonstrates that cells receiving TAT-functionalized mitochondria, either as free mitochondria or released from hydrogel microparticles, may exhibit higher differentiation scores and fusion indices compared to control conditions. This enhanced differentiation may suggest improved myogenic potential in the recipient muscle cells.

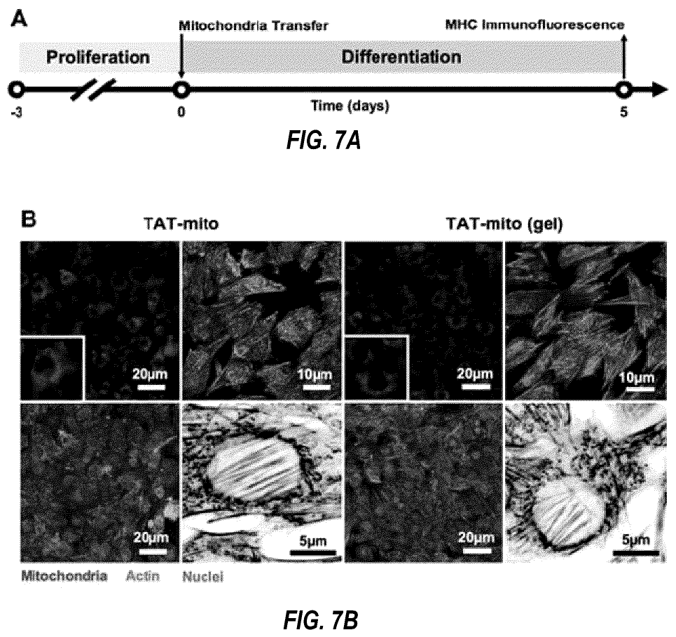

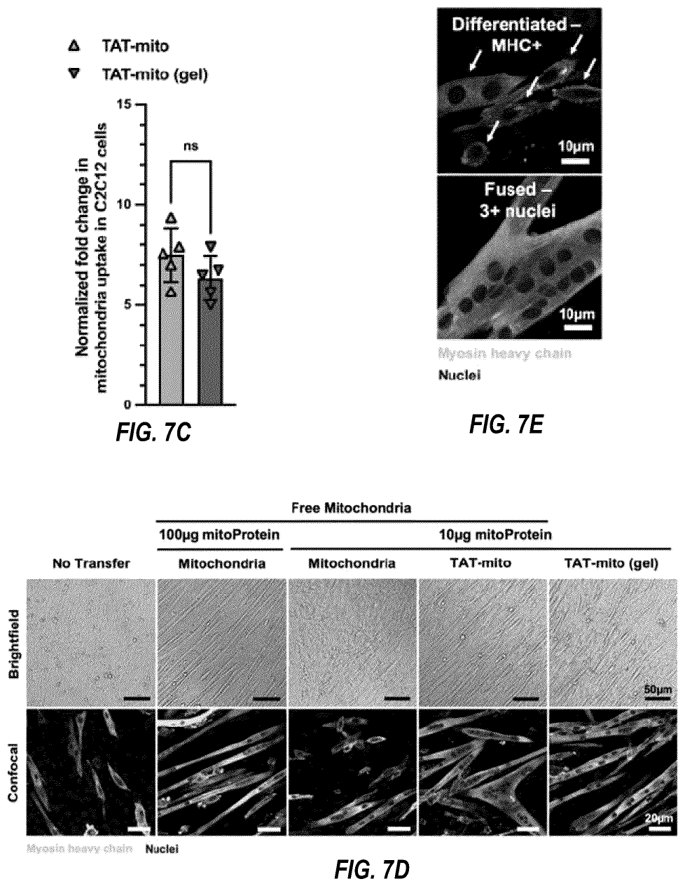

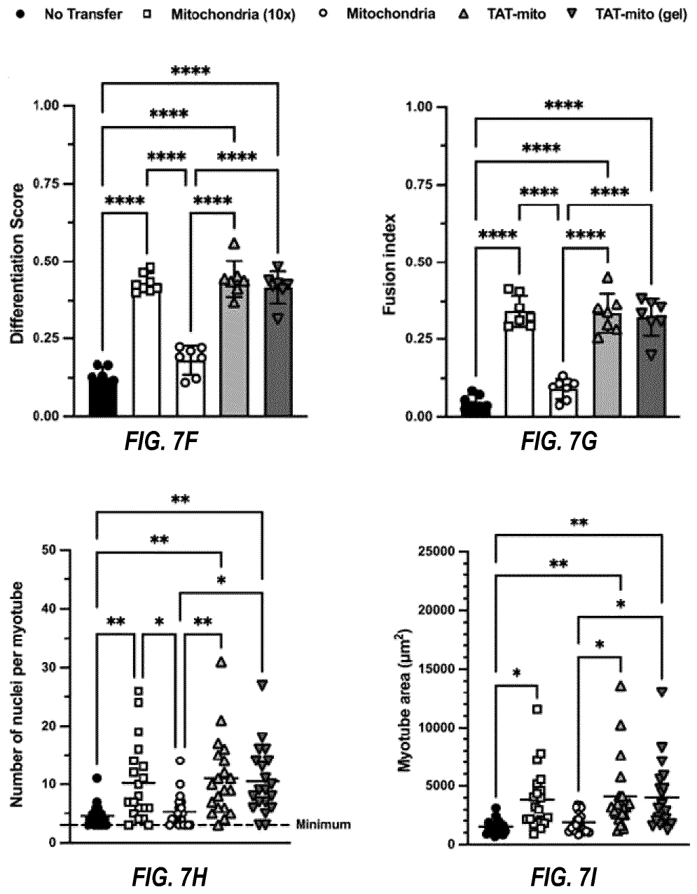

FIGS. 7F-7I provide additional data on differentiation metrics for muscle cells under various treatment conditions. The graphs show measurements such as differentiation score, fusion index, nuclei per myotube, and myotube area. In some cases, cells receiving TAT-functionalized mitochondria may demonstrate improved performance across these metrics compared to control conditions. These results may indicate enhanced muscle cell differentiation and fusion following mitochondrial transfer.

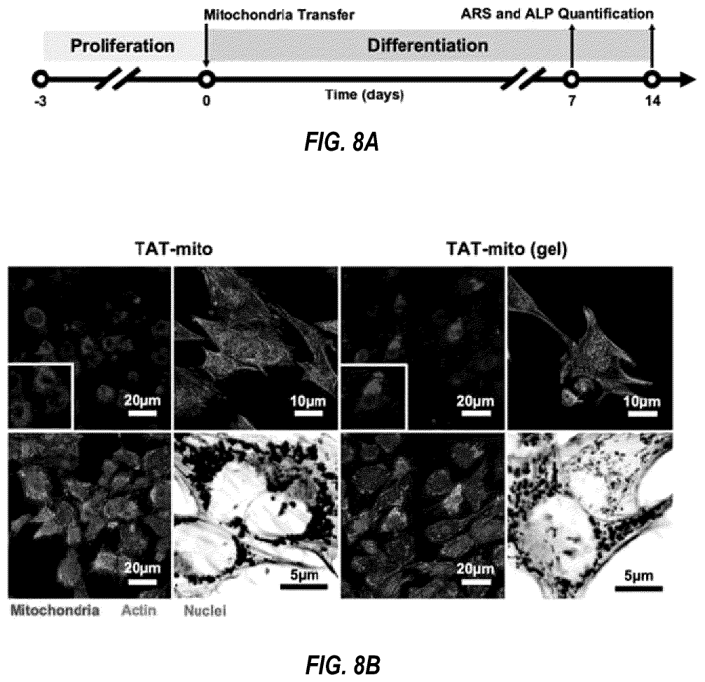

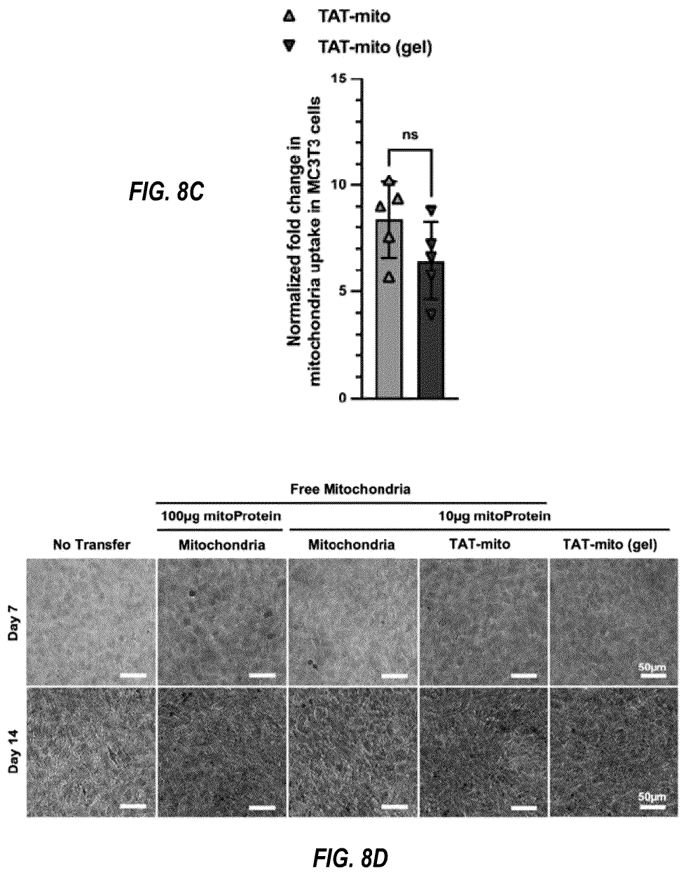

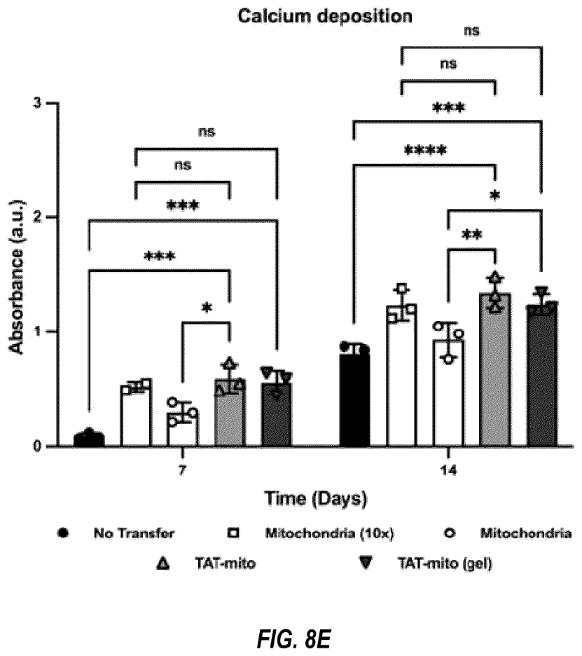

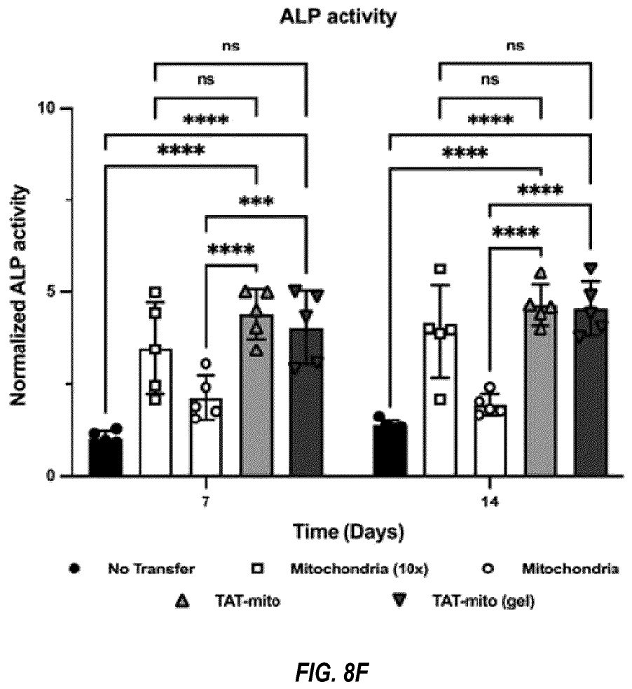

The systems and methods described herein may also have applications in bone regeneration. FIG. 1E and FIG. 8E show absorbance measurements related to calcium deposition in bone-forming cells under different experimental conditions. The graphs demonstrate that cells receiving TAT-functionalized mitochondria may exhibit increased calcium deposition over time compared to control conditions. This increased calcium deposition may suggest enhanced osteogenic differentiation in the recipient cells.

FIG. 8F depicts alkaline phosphatase (ALP) activity measurements in bone-forming cells. The graph shows that cells receiving TAT-functionalized mitochondria may display higher ALP activity compared to control conditions. Increased ALP activity may be associated with enhanced osteogenic differentiation and bone formation potential.

In some cases, the hydrogel microparticles containing functionalized mitochondria may be administered to a subject for treating diseases or injuries. The administration may involve injecting the hydrogel into a site of disease or injury. This localized delivery approach may allow for targeted mitochondrial transfer to specific tissues or cell populations.

The hydrogel may be administered in combination with a cell therapy. In some cases, the cell therapy may comprise mesenchymal stem cells. The combination of mitochondrial transfer and stem cell therapy may potentially enhance tissue regeneration processes in the treated area.

The systems and methods described herein may have applications in treating musculoskeletal diseases or injuries. The enhanced differentiation observed in muscle and bone-forming cells following mitochondrial transfer may contribute to improved tissue regeneration in these contexts. The ability to deliver mitochondria in a controlled manner through enzyme-responsive hydrogel microparticles may allow for targeted and sustained mitochondrial transfer to support tissue healing and regeneration processes.

The systems and methods described herein may integrate multiple components to achieve on-demand delivery of mitochondria. FIG. 1A illustrates a system diagram, including functionalized mitochondria and enzyme-responsive hydrogel microparticles.

The process may begin with surface modification of mitochondria using a composition comprising a polymer backbone conjugated with TAT peptide and triphenylphosphine moieties. This surface modification may enhance the cellular uptake capabilities of the mitochondria.

The functionalized mitochondria may then be encapsulated within enzyme-responsive hydrogel microparticles. As shown in FIG. 1A, these microparticles may comprise a crosslinked polymer network containing enzyme-cleavable peptide sequences.

In some cases, the hydrogel microparticles containing functionalized mitochondria may be administered to a subject for treating diseases or injuries. The hydrogel may be designed to react with specific enzymes present at the treatment site.

Matrix metalloproteinases (MMPs) may be produced by the subject as a result of disease or injury processes. In some cases, these MMPs may interact with the enzyme-cleavable peptides in the hydrogel structure. This interaction may cause degradation of at least a portion of the hydrogel.

The degradation of the hydrogel in response to MMPs may result in the release of the encapsulated mitochondria. FIG. 1B demonstrates this controlled release process, showing cumulative mitochondria release over time in the presence of collagenase, an enzyme that may mimic MMP activity.

Following release from the hydrogel microparticles, the functionalized mitochondria may be taken up by cells in the surrounding tissue. The surface modifications on the mitochondria may facilitate enhanced cellular uptake through TAT peptide-mediated endocytosis.

Once internalized by recipient cells, the transferred mitochondria may contribute to various biological effects. In some cases, these effects may include increased cellular energy production, enhanced differentiation processes, or improved tissue regeneration capabilities.

The integration of mitochondrial surface modification, enzyme-responsive hydrogel encapsulation, and controlled release mechanisms may allow for targeted and on-demand delivery of mitochondria to specific tissues or cell populations. This system may provide a means for harnessing the potential therapeutic benefits of mitochondrial transfer in various disease or injury contexts.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A composition for modifying the surface of mitochondria, comprising:
a polymer backbone;
a first chemical moiety conjugated to the polymer backbone, the chemical moiety comprising a peptide according to SEQ ID NO: 1; and
a second chemical moiety conjugated to the polymer backbone, the second chemical moiety comprising triphenylphosphine.

2. The composition of claim 1, wherein the polymer backbone is an inert polymer backbone.

3. The composition of claim 1, wherein the polymer backbone is a polysaccharide.

4. The composition of claim 3, wherein the polysaccharide is a dextran.

5. A hydrogel, comprising:
a plurality of hydrogel particles, each of the hydrogel particles comprising:
a first polymer crosslinked with an enzyme cleavable peptide to form a crosslinked polymer; and
at least one mitochondria,
wherein each hydrogel particle in the plurality of hydrogel particles has an average diameter no greater than 100 microns.

6. The hydrogel of claim 5, wherein the first polymer is polyethylene glycol.

7. The hydrogel of claim 5, wherein the enzyme cleavable peptide is selected from the group consisting of: SEQ ID NO: 2; SEQ ID NO: 3; and SEQ ID NO: 4.

8. The hydrogel of claim 5, further comprising a second polymer.

9. The hydrogel of claim 8, wherein the second polymer is poly(ethylene glycol) diacrylate (PEGDA).

10. The hydrogel of claim 8, wherein a ratio of the second polymer to the crosslinked polymer is no greater than 1:1 mol %.

11. The hydrogel of claim 5, wherein a surface of the at least one mitochondria is modified with the composition of claim 1.

12. The hydrogel of claim 5, wherein a surface of the at least one mitochondria is modified with the composition of claim 3.

13. The hydrogel of claim 5, wherein a surface of the at least one mitochondria is modified with the composition of claim 4.

14. A method of treating a disease and/or injury in a subject, comprising:
administering, to the subject, the hydrogel of claim 5.

15. The method of claim 14, wherein the disease and/or injury is a musculoskeletal disease or injury.

16. The method of claim 15, wherein the musculoskeletal disease or injury is selected from the group consisting of: muscle injury, bone injury, and cartilage injury.

17. The method of claim 14, wherein administering comprises injecting the hydrogel into a site of the disease and/or injury.

18. The method of claim 14, wherein the hydrogel is administered in combination with a cell therapy.

19. The method of claim 18, wherein the cell therapy comprises tissue-derived or blood-derived cells.

20. The method of claim 14, wherein subsequent to administering, to the subject, the hydrogel, the hydrogel reacts with matrix metalloproteinases produced by the subject as a result of the disease and/or injury to cause degradation of at least a portion of the hydrogel and release of the at least one mitochondria.
